(19) United States
(12) Patent Application Publication
(10) Pub. No.: US 2003/0108944 A1
(43) Pub. Date: Jun. 12, 2003

(54) COMBINATORIAL PREPARATIONS OF PHOSPHORUS-CONTAINING ACTIVE COMPOUNDS AND INTERMEDIATES BY SOLID PHASE SYNTHESIS

(76) Inventors: KLAUS HAAF, KELKHEIM (DE); MARCEL PATEK, TUCSON, AZ (US)

Correspondence Address: FROMMER LAWRENCE & HAUG, 745 FIFTH AVENUE- 10TH FL., NEW YORK, NY, 10151, US

(21) Appl. No.: 09/167,351

(22) Filed: Oct. 7, 1998

Related U.S. Application Data
(60) Provisional application No. 60/061,619, filed on Oct. 9, 1997.

(30) Foreign Application Priority Data
Oct. 10, 1997 (DE) 197 45 628.6

(51) Int. Cl. G01N 33/53; C07F 9/02; C12P 7/62

(52) U.S. Cl. 435/7.1; 558/87

(57) ABSTRACT

The invention relates to solid phase processes for the systematic preparation of chemical compounds from the group of the phosphonous or phosphinic acids and/or derivatives thereof and the corresponding substance libraries which can be employed for test purposes, in particular tests for biological activity. The compounds (I)

YR¹P(═O)(OR³)R² (I)

in which Y, R¹, R², R³ are as defined in claim 1 are prepared by reacting a resin-linker adduct (II)

[resin polymer]-[linker-Z-E¹-S¹]ₙ

in the presence of a suitable Pd catalyst with a phosphinate (III)

A¹-O—(PHO)A^{□} (III)

with substitution of the group S¹ to give the compound (IV)

[resin polymer]-[linker-Z-E¹-P(H)(═O)—OA¹ (IV)

and cleaving the compound (I) after derivatization reactions on the resin from the resin-linker adduct.

The invention also provides the intermediate steps and resin-linked intermediate compounds, and also the substance libraries obtained.

The invention relates to the technical field of the synthesis of chemical compounds having certain common structural features, in particular in the field of the intermediates and active compounds from the group of the phosphonous acids and phosphinic acids and esters thereof.

Phosphorus-containing compounds are frequently encountered in the metabolism of animal and plant organisms. Using generally known examples, it has already been shown that structural variations of such compounds can be active compounds in the field of the medicaments or crop protection agents. However, it is a growing problem to discover, from the large number of structural variations of potential active compounds, those having the desired properties. The increasing demands on the properties of novel biologically active substances for crop protection or medicine mean that the development of an active compound which is ready for marketing is associated with the preparation and testing of an increasingly large numbers of test substances. In the estimation of many specialists, this tendency will probably persist despite improved knowledge about the biochemistry of known active compounds and computer-assisted calculations of molecular structures and properties (“molecular modeling”). In order not to allow the expense and consumption of time to increase equally, the object for research into novel active compounds is to develop more effective methods for the preparation of large numbers of novel or systematically varied test compounds.

The methods for the systematic preparation of large numbers of test compounds and especially methods suitable for their analysis and biological examination are summarized under the term “combinatorial chemistry”; cf. for example J. S. Früchtel, G. Jung in Angew. Chem. 108 (1996) 1946 or Angew. Chem. Int. Ed. Engl. 35 (1996), pp. 17-42.

Some combinatorial synthesis methods are aimed at preparing jointly (“in a pool”), in a manner which is as effective and standardized as possible, a large number of structurally variant compounds in as few reaction steps as possible and jointly testing them for biological activity; cf. for example the divide, couple and recombine method according to a) K. S. Lam, S. E. Salmon, E. M. Hersh, V. J. Hruby, W. M. Kazmeiersky, R. J. Knapp in Nature 82 (1991) 354, b) A. Furka, F. Sebestyen, M. Asgedom, G. Dibo in Int. J. Pept. Protein Res. 37 (1991) 487. If an entire group of compounds (“pool”) contains no active compound, a single joint test suffices in principle to exclude these structural variants. If the joint test, however, indicates activity, the variation in the preparation of the test compounds can be decreased in a controlled manner in order to limit the group containing the active compound or the active compounds and finally to determine the structure of the active compounds.

Generally, however, the “pooling” method described can no longer be used efficiently when it concerns the optimization of active compound structures and many similarly active compounds are present or expected in the group of test compounds or else when large amounts of the compounds are needed for the first test.

To achieve the last-mentioned object, the starting compound used is often a compound with known biological activity, the so-called lead structure or lead compound, and the structure of the lead compound is varied systematically with the aid of a preparation process which is standardized to a great extent (“combinatorial preparation process”), by use of a large number of different starting materials. The individual compounds prepared in each case are then tested individually for their biological activity in order to find the optimally active compound with the same type of action.

The known synthesis methods from combinatorial chemistry (see J. S. Früchtel, G. Jung in Angew. Chem. 108 (1996) 19-46 and the literature cited there) include a group of methods in which the respective active compound is prepared stepwise bound to a solid, in particular bound to an organic polymer (hereinbelow referred to as “synthetic or natural resin”, “resin” or “resin polymer”).

With the aid of the binding to the solid, for example to the resin in the form of particles of large particle size or spheres, the intermediates are in principle handleable macroscopically. The synthesis of an active compound via several reaction steps then needs less expenditure on isolation and purification than in conventional methods, because these steps generally can be effected in the form of simple filtration and washing of the resinous substances. The resin-bound finished active compound molecule must finally be cleaved again from the resin.

In the choice of suitable resin-molecule systems, problems fundamentally result due to the conflict of aims both in ensuring a desired high stability of the bond between moieties synthesized and the resin when using different reaction types and conditions and in making possible a gentle method for the predominant or complete cleavage of the finished synthesis product from the resin.

The invention is based on the object of making available a combinatorial synthesis method based on resin-bound synthesis building blocks and products, which allows the synthesis of a wide variation of potentially biologically active compounds containing a phosphorus atom and having similar partial structure.

The invention relates to a process using intermediates which are linked to a resin polymer for preparing chemical compounds of the formula (I)

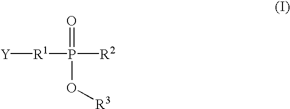

in which

| | |
|---|---|
| R¹ | is an unsubstituted or substituted aromatic or heteroaromatic radical, |
| R² | is hydrogen or an organic radical which may be linked to the rest of |
| | the compound of formula (I) via hetero atoms, |
| R³ | is hydrogen or an organic radical which may be linked to the rest of |
| | the compound of the formula (I) via hetero atoms and |
| Y | is the functional group which is formed at the molecule of the |
| | formula (I) after the compound (I) has been cleaved off from the |
| | resin polymer, |

which comprises

a) reacting a resin-linker adduct of the formula (II)

[resin polymer]-[linker-Z-E¹-S¹]ₙ (II)

in which

| | |
|---|---|
| [resin polymer] | is the radical of a resin which, in the resin-linker |
| | compound (II), is connected via n binding sites with the n |
| | groups of the formula [linker-Z-E¹-S¹], |
| linker | is in each case an organic linker, |
| Z | is a linker-specific functional group or bond which, after |
| | cleavage of the compound (I) from the resin polymer- |
| | linker radical, gives rise to the group Y in formula (I), |
| E¹ | is defined as R¹ in formula (I) or is a radical which is |
| | suitable for preparing R¹ in compound (I), |
| S¹ | is a functional group suitable for palladium-catalyzed |
| | substitutions analogous to the Heck reaction, |
| n | is the number of the functional groups [linker-Z-E¹-S¹] at |
| | the resin, which depends on the molecular weight of the |
| | resin polymer and is greater than or equal to 1, |

in the presence of a suitable palladium catalyst with a compound selected from the group of the phosphinates (derivatives of hypophosphoric acid) of the formula (III)

A¹-O—(PHO)A* (III)

in which

| | |
|---|---|
| A¹ | is hydrogen or an organic radical, preferably the radical of the |
| | formula R³ in formula (I) or an alkyl radical which differs therefrom, |
| | in particular (C₁-C₄)alkyl, or a trialkylsilyl radical such as |
| | trimethylsilyl (TMS) and |
| A* | is a group which can be removed hydrolytically or after an inter- |
| | mediate reaction, for example an alkyl group, a trialkylsilyl radical |
| | such as TMS, or dialkoxymethyl, |

preferably with a compound of the formula (III-1), (III-2) or (III-3)

H₂P(═O)—O-A¹ (III-1)

A¹-O—PH—O-A* (III-2)

A¹-O—P(H)(═O)(A*) (III-3)

in which A¹ and A* are as defined above,

in particular with a phosphinate derivative of the formula

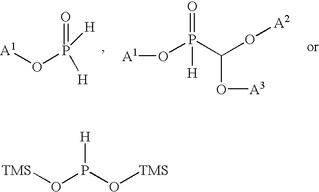

in which A¹ is an organic radical, preferably the radical of the formula R³ in formula (1) or an alkyl radical which differs therefrom, in particular (C₁-C₄)alkyl, A² and A³ are each an alkyl radical such as (C₁-C₄)alkyl and TMS=trimethylsilyl,

with substitution of the group S¹ to give a resin-bound compound of the formula (IV)

[resin polymer]-[linker-Z-E¹-P(H)(═O)—O-A¹]ₙ (IV)

in which A¹ is as defined in formula (III), and

b) derivatizing—preferably in the case where E¹ in the compound (II) employed in a) is not R¹—if appropriate, the compound (IV) in one or more further reaction steps at the organic radical E¹ to give the radical (E¹)′, thus yielding one or more resin-bound intermediates of the formula (IV)′

[resin polymer]-[linker-Z-(E¹)-P(H)(═O)—O-A¹]ₙ (IV)′

in which A¹ is as defined in formula (III), and

c) hydrolyzing, if appropriate, the compound of the formula (IV) or (IV)′ from step a) or b) to give a compound (V) or (V)′ suitable, i.e. in particular suitable with regard to the reactivity of the linker bond and the swelling capacity of the resin employed in each case, for the resin-bound synthesis

[resin polymer]-[linker-Z-E¹-P(H)(═O)—OH]ₙ (V)

[resin polymer]-[linker-Z-(E¹)′—P(H)(═O)—OH]ₙ (V)′

and

d) esterifying, if appropriate, the compound (V) or (V)′ obtained according to c) to give the compound of the formula (VI) or (VI)′

[resin polymer]-[linker-Z-E¹-P(H)(═O)—O—R³]ₙ (VI)

[resin polymer]-[linker-Z-(E¹)′—P(H)(═O)—O—R³]ₙ (VI)′

in which

R³ is defined as R³ in formula (I), but is not hydrogen, and

e) reacting, if appropriate, a compound (IV), (V) or (VI) or (IV)′, (V)′ or (VI)′ obtained according to a), b), c) or d), whose common structural feature is the phosphonous acid or phosphonous ester group, forming a phosphorus-carbon bond, to give compounds of the formulae (VII) or (VIII) or (VII)′ or (VIII)′

[resin polymer]-[linker-Z-E¹-P(R²)(═O)—O-A⁴]ₙ (VII)

[resin polymer]-[linker-Z-(E¹)′—P(R²)(═O)—O-A⁴]ₙ (VII)′

[resin polymer]-[linker-Z-E¹-P(E²)(═O)—O-A⁴]ₙ (VIII)

[resin polymer]-[linker-Z-(E¹)′—P(E²)(═O)—O-A⁴]ₙ (VIII)′

in which

| | |
|---|---|
| R² | is as defined in formula (I), |
| E² | is an organic radical which can be derivatized to the radical R², |
| A⁴ = | A¹, H or R³, and |

f) modifying the compounds obtained according to the abovementioned steps if required at the radicals E¹, (E¹)′, E² and A⁴ in such a manner that the resin-bound compound of the formula (IX) is obtained

[resin polymer]-[linker-Z-R¹—P(R²)(═O)—O—R³]ₙ (IX)

in which R¹, R², R³ are as defined in formula (I), and

g) cleaving the compound of the formula (I) from the resin-linker adduct of the formula (IX), where in the formulae (IV) to (IX) and (IV)′ to (VIII)′ the radicals [resin polymer], linker, Z are as defined in formula (II) and E¹ or (E¹)′ in the formulae (V) to (VIII) or (V)′ to (VIII)′ are as defined in formula (IV) or (IV)′.

The invention also relates to the individual steps of the process according to the invention and to the novel compounds of the formula (I), (II) and (IV) to (IX) and (IV)′ to (VIII)′.

A particular aspect of the invention is the wide range of structural variations of the compounds of the formula (I) which can be prepared. This is possible primarily because a large number of derivatization reactions, which can be carried out with high yields in the individual steps, are suitable for the phosphonous acid or phosphonous ester groups introduced in step a). It is particularly surprising that the introduction of the phosphorus-containing groups on the resin skeleton succeeds with good yield. From experience, many of the reactions known from free-solution chemistry do not succeed under analogous conditions, do not succeed with good yields or do not succeed at all when one of the reaction components has been fixed on carriers. In solid phase syntheses, the introduction of phosphonous acid or phosphonous ester groups under the conditions of the palladium-catalyzed Heck reaction was hitherto unknown.

Likewise surprising is the good yield and purity of the end products (I) which, owing to their functional groups, are very polar molecules. In contrast, some corresponding syntheses in free solution result in extreme loss of yield.

In the formula (I) and the other formulae (II) to (VII), an organic radical is a carbon-containing radical, for example a (hetero)aromatic radical with or without substitution or an aliphatic, i.e. nonaromatic, organic radical which, apart from carbon atoms and hydrogen atoms, can also contain hetero atoms, or a corresponding araliphatic radical.

The size of the suitable organic radicals may vary considerably; an organic radical including possibly contained substituents preferably contains less than 30 carbon atoms, in particular 1 to 20 carbon atoms, and preference is generally given to smaller radicals having 1 to 12 carbon atoms. Possible substituents of an organic radical are likewise (hetero)aromatic and aliphatic radicals, including functional groups, the functional groups preferably being highly compatible with the functional groups otherwise present in the fixed moiety. For example, as functional groups, no oxidative groups should be present if the linker is sensitive to oxidation and thus would react even under the conditions of the combinatorial synthesis.

Organic radicals are, for example, optionally substituted hydrocarbon radicals or hydrocarbonyloxy radicals. A hydrocarbon radical is a straight-chain, branched or cyclic and saturated or unsaturated or aromatic hydrocarbon radical; for example alkyl, alkynyl, cycloalkyl, cycloalkenyl or aryl; a hydrocarbon radical is preferably alkyl, alkenyl or alkynyl having up to 12 carbon atoms or cycloalkyl having 3, 4, 5, 6, 7 or 8 ring atoms or aryl; the same applies correspondingly to a hydrocarbon radical in a hydrocarbonyloxy radical. Organic radicals which may be attached via a hetero atom include groups such as trialkylsilyl, for example trimethylsilyl (TMS).

Aryl is a mono-, bi- or polycyclic, carbocyclic aromatic ring system; in the case of substitution, or more precisely in the case of cyclic substitution, bicyclic or polycyclic ring systems having at least one aromatic ring which is fused to one or more cycloaliphatic, optionally partially unsaturated rings, are in particular also included. Optionally cyclically substituted aryl is, for example, phenyl, naphthyl, tetrahydronaphthyl, indenyl, indanyl, pentalenyl, fluorenyl and the like, it being possible for the ring systems mentioned to be additionally further substituted in the case of general substitution; preferably aryl is an unsubstituted phenyl or naphthyl ring; substituted aryl is preferably a phenyl radical which is unsubstituted or substituted, the substituents not being fused rings.

Heteroaryl or a heteroaromatic radical is a mono-, bi- or polycyclic aromatic ring system in which at least 1 ring contains one or more hetero atoms, for example pyridyl, pyrimidinyl, pyridazinyl, pyrazinyl, triazinyl, thienyl, thiazolyl, oxazolyl, isoxazolyl, furyl, pyrrolyl, pyrazolyl and imidazolyl. In the case of substitution, bicyclic or polycyclic aromatic or benzo-fused compounds or compounds fused with cycloaliphatic rings, for example quinolinyl, benzoxazolyl and the like, are also particularly included. Heteroaryl also includes a heteroaromatic ring which is preferably 5- or 6-membered and contains 1, 2 or 3 hetero ring atoms, in particular selected from the group consisting of N, O and S.

A heterocyclic radical (heterocyclyl) or ring (heterocycle) can be saturated, unsaturated or heteroaromatic (heteroaryl); it contains one or more hetero ring atoms, preferably selected from the group consisting of N, O and S; it is preferably a non-aromatic ring having 3 to 8 ring atoms and 1 to 3 hetero ring atoms selected from the group consisting of N, O and S or it is a heteroaromatic ring having 5 or 6 ring atoms and contains 1, 2 or 3 hetero ring atoms selected from the group consisting of N, O and S. The radical can be, for example, a heteroaromatic radical or ring as defined above, or it is a partially hydrogenated radical such as oxiranyl, pyrrolidyl, piperidyl, piperazinyl, dioxolanyl, morpholinyl, tetrahydrofuryl. Substituents which are suitable for a substituted heterocyclic radical are the substituents mentioned further below, and additionally also oxo. The oxo group can also be present on the hetero ring atoms, which can exist at various oxidation levels, for example in the case of N and S.

Substituted radicals, such as substituted hydrocarbon radicals, for example substituted alkyl, alkenyl, alkynyl, aryl, phenyl and benzyl, or substituted heteroaryl, a substituted bicyclic radical with or without aromatic moieties, are, for example, a substituted radical derived from the unsubstituted skeleton, the substituents being, for example, one or more, preferably 1, 2 or 3, radicals selected from the group consisting of halogen, alkoxy, haloalkoxy, alkylthio, hydroxyl, amino, nitro, cyano, azido, alkoxycarbonyl, alkylcarbonyl, formyl, carbamoyl, mono- and dialkylaminocarbonyl, substituted amino, such as acylamino, mono- or dialkylamino, and alkylsulfinyl, haloalkylsulfinyl, alkylsulfonyl, haloalkylsulfonyl and, in the case of cyclic radicals, also alkyl and haloalkyl, and unsaturated aliphatic radicals which correspond to the abovementioned saturated hydrocarbon-containing radicals, such as alkenyl, alkynyl, alkenyloxy, alkynyloxy and the like. Preferred among the radicals having carbon atoms are those having 1 to 4 carbon atoms, in particular 1 or 2 carbon atoms. Preferred substituents are generally selected from the group consisting of halogen, for example fluorine and chlorine, C₁-C₄-alkyl, preferably methyl or ethyl, C₁-C₄-haloalkyl, preferably trifluoromethyl, C₁-C₄-alkoxy, preferably methoxy or ethoxy, C₁-C₄-haloalkoxy, nitro and cyano. Especially preferred are the substituents methyl, methoxy and chlorine.

Unsubstituted or substituted phenyl is preferably phenyl which is unsubstituted or mono- or polysubstituted, preferably up to trisubstituted, by identical or different radicals selected from the group consisting of halogen, C₁-C₄-alkyl, C₁-C₄-alkoxy, C₁-C₄-haloalkyl, C₁-C₄-haloalkoxy und nitro, for example o-, m- and p-tolyl, dimethylphenyl radicals, 2-, 3- and 4-chlorophenyl, 2-, 3- and 4-trifluoro- and -trichlorophenyl, 2,4-, 3,5-, 2,5- and 2,3-dichlorophenyl, o-, m- and p-methoxyphenyl.

The radicals alkyl, alkoxy, haloalkyl, haloalkoxy, alkylamino and alkylthio and the corresponding unsaturated and/or substituted radicals are in each case straight-chain or branched in the carbon skeleton. Unless specifically mentioned, the lower carbon skeletons, for example those having 1 to 4 carbon atoms or, in the case of unsaturated groups, 2 to 4 carbon atoms, are preferred for these radicals. Alkyl radicals, also in the composite meanings such as alkoxy, haloalkyl and the like, are, for example, methyl, ethyl, n- or i-propyl, n-, i-, t- or 2-butyl, pentyl radicals, hexyl radicals such as n-hexyl, i-hexyl and 1,3-dimethylbutyl, heptyl radicals, such as n-heptyl, 1-methylhexyl and 1,4-dimethylpentyl. Cycloalkyl is a cycloaliphatic hydrocarbon radical such as cyclopropyl, cyclobutyl, cyclopentyl or cyclohexyl and the like; alkenyl, cycloalkenyl and alkynyl have the meaning of the unsaturated radicals which are possible and which correspond to the alkyl radicals; alkenyl is, for example, allyl, 1-methylprop-2-en-1-yl, 2-methylprop-2-en-1-yl, but-2-en-1-yl, but-3-en-1-yl, methylbut-3-en-1-yl and 1-methylbut-2-en-1-yl; cycloalkenyl is, for example, cyclopentenyl and cyclohexenyl; alkynyl is, for example, propargyl, but-2-yn-1-yl, but-3-yn-1-yl or 1-methylbut-3-yn-1-yl.

Alkenyl in the form “(C₃-C₄)alkenyl” or “(C₃-C₆)alkenyl” is preferably an alkenyl radical having 3 to 4 and 3 to 6 carbon atoms, respectively, where the double bond is not located on the carbon atom attached to the remaining moiety of the compound (“yl” position). The same applies analogously to (C₃-C₄)alkynyl and the like.

Halogen is, for example, fluorine, chlorine, bromine or iodine, haloalkyl, haloalkenyl and haloalkynyl are alkyl, alkenyl and alkynyl, respectively, which are fully or partially substituted by halogen, preferably by fluorine, chlorine and/or bromine, in particular by fluorine or chlorine, such as CFhd 3, CHF₂, CH₂F, CF₃CF₂, CH₂FCHCl₂, CCl₃, CHCl₂, CH₂CH₂Cl; haloalkyl is, for example, OCF₃, OCHF₂, OCH₂F, CF₃CF₂O, OCH₂CF₃ and OCH₂CH₂Cl; the same applies analogously to haloalkenyl and other halogen-substituted radicals.

Mono- or disubstituted amino is a chemically stable radical selected from the group consisting of the substituted amino radicals which are N-substituted, for example, by one or two identical or different radicals selected from the group consisting of alkyl, alkoxy, acyl and aryl; preferably monoalkylamino, dialkylamino, acylamino, arylamino, N-alkyl-N-arylamino and N-heterocycles; preferred are alkyl radicals having 1 to 4 carbon atoms; aryl is preferably phenyl or substituted phenyl; acyl is defined as indicated further below and is preferably (C₁-C₄)alkanoyl. The same applies analogously to substituted hydroxylamino or hydrazino.

An acyl radical is the radical of an organic acid, for example the radical of a carboxylic acid and radicals of acids derived therefrom, such as thiocarboxylic acid, iminocarboxylic acids with or without N-substitution, or the radical of carbonic monoesters, carbamic acid with or without N-substitution, sulfonic acids, sulfinic acids, phosphonic acids, phosphinic acids. Acyl is, for example, formyl, alkylcarbonyl such as [(C₁-C₄)-alkyl]carbonyl, phenylcarbonyl where the phenyl ring may be substituted, for example as shown above for phenyl, or alkyloxycarbonyl, phenyloxycarbonyl, benzyloxycarbonyl, alkylsulfonyl, alkylsulfinyl, N-alkyl-1-iminoalkyl and other radicals of organic acids.

The formulae also embrace stereoisomers containing, for example one or more asymmetric carbon atoms or else double bonds which are not specifically indicated in the respective formulae. The stereoisomers of the same chemical linkage which are possible and which are defined by their specific spatial form, such as enantiomers, diastereomers, Z isomers and E isomers, are therefore all embraced by the formula and can be obtained from stereoisomer mixtures by customary methods, or else be prepared by stereoselective reactions in combination with the use of stereochemically pure starting materials.

The formulae also embrace tautomers of the indicated compounds, as far as they are formed by proton migration and as far as they are chemically stable tautomers.

Many of the compounds of the formula (I) can form salts, for example those where in the case of R³═H the hydrogen of the group —P(═O)(R²)(OH) or else other acidic hydrogen atoms which are present (for example from carboxyl groups, inter alia) are replaced by an agriculturally suitable cation. These salts are, for example, metal salts; preferably alkali metal or alkaline earth metal salts, in particular sodium salts and potassium salts, or else ammonium salts or salts with organic amines. Salt formation can also take place by addition of an acid to basic groups, such as, for example, amino and alkylamino. Suitable acids for this purpose are strong inorganic and organic acids, for example HCl, HBr, H₂SO₄ or HNO₃.

The organic linker in the compounds of the formulae (II) and (IV) to (IX) has the function of a bridge between the resin polymer and the part of the molecule which is to be structurally modified. The linker must make possible the binding of the part of the molecule mentioned and its later removal. The linker must additionally be able to be applied to the resin polymer, generally by means of a chemical reaction if the resin polymer cannot be synthesized from suitable monomers which contain the linker. It may be possible to do entirely without a structurally specific linker; in this case, the linker is a direct bond.

Suitable linkers are structurally very different radicals which, depending on the binding sites on the resin polymer, have to have suitable binding sites and functional groups, and usually a resin-linker compound of the formula (X)

[resin polymer]-[linker-X]ₙ (X)

where [resin polymer], linker and n are as defined in the abovementioned formula (II) and X is a functional group which is specific for the linker used in each case, is first prepared or made available. The resin-linker compound (X) is then reacted with an aromatic or heteroaromatic compound (“scaffold system”) of the formula (XI)

Y¹-E¹-S¹ (XI)

where E¹ and S¹ are defined as in the abovementioned formula (II) and Y¹ is a functional group which reacts with the functional group X of the resin-linker compound to give the bridge Z, similar to known methods to give the resin-linker adduct of the formula (II).

Suitable for the process are structurally very different linkers including, for example, those which can also be employed from resin-bound synthesis for the binding of carboxylic acids, for example of amino acids, in peptide synthesis.

Compounds (linker components) which can be employed for the synthesis of the linker in combination with a resin containing amino groups, for example an aminomethylenepolystyrene resin, or a resin containing hydroxyl groups, are, for example, linker components having a carboxylic acid group. The preparation of the resin-linker compound of the formula (X) is then carried out in each case by reaction of the carboxyl group of a linker component (XII) with an amino group or hydroxyl group of the resin (amide formation or ester formation).

In some cases, the linkers can also be prepared stepwise; in a first step a carboxylic acid is condensed with the resin containing amino acids and the resulting modified resin is further modified on the introduced groups as far as the desired resin-linker compound.

In addition, resin-linker compounds are known which are synthesized on the basis of further resins and in another manner. Examples of linker components (XII) and resin-linker compounds of the formula (X) are listed below in Tables 1 and 2; a linker component is in each case the compound of the formula (XII)

W-linker-X (XII)

where W is the leaving group or functional group to be activated to give the leaving group, which is replaced in the reaction with the functional group of the resin, for example the amino group or hydroxyl group of the resin; in the case where the resin-linker compound (X) is prepared differently or the preparation is not given in detail, the radical W=polymer or [symbol] is given, indicating the binding site of the functional group linker-X on the resin polymer; X is as defined further above; literature references: see J. S. Früchtel, G. Jung, Angew. Chem. 108 (1996) 1946 and literature cited therein:

TABLE 1
Base-stable linker groups between substrate and solid phase

| W-linker-X | Notes |
|---|---|
| 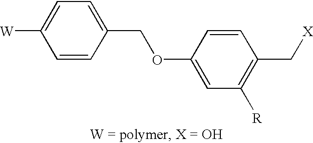 | a) Wang linker (R = H); suitable for the fixation of carboxylic acids; cleavage with 95% strength trifluoroacetic acid (TFA) b) SASRIN linker; (R = OMe); fixation of carboxylic acids; cleavage with 1% strength TFA |
| 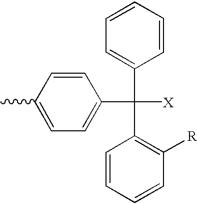 | a) Tritylchloride linker (R = H, X = Cl); fixation of nucleophiles, cleavagewith weak acids (HOAc); b) 2-Chlorotritylchloride linker (R = Cl, X = Cl), fixation of nucleophiles, cleavage with very weak acids such as HOAc/CH₂Cl₂(1/4) |
| 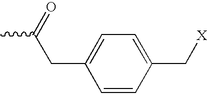 | PAM linker; fixation of carboxylic acids, cleavage with HF, TFMSA; |
| 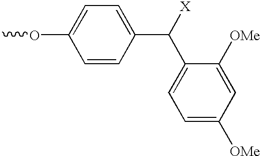 | a) Rink acid (X = OH); Fixation of carboxylic acids cleavage with HOAc/CH₂Cl₂b) Rink amide (X = NH-Fmoc); fixation of carboxylic acids as amide, cleavage with TFA/CH₂Cl₂ |
| 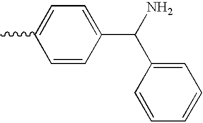 | BHA anchor; fixation of carboxylic acids; cleavage with HF, TFMSA |
| 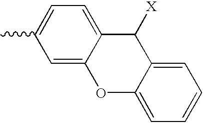 | Sieber amide; fixation of carboxylic Acids; cleavage with TFA/CH₂Cl₂ (1/99) Fmoc = 9-fluorenylmethoxy- X = NH-Fmoc-carbonyl |

TABLE 2
Acid-stable linker groups between substrate and solid phase

| W-linker-X | Notes |
|---|---|
| 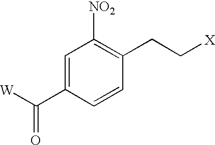 | Fixation of carboxylic acids Cleavage with DBU/piperidine (β-elimination); W = OH X = OH |
| 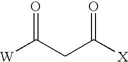 | Fixation of alcohols, amines; cleavage with NaOH (hydrolysis); W = OH, X = OH |
| 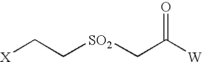 | Fixation of carboxylic acids; cleavage with Bu₄NF W = OH, X = OH |
| 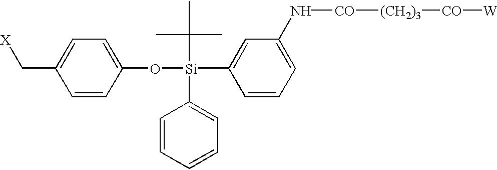 | W = OH, X = OH Fixation of carboxylic acid; cleavage with BU₄NH; |
| 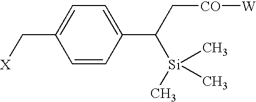 | Fixation of carboxylic acids; Cleavage with Bu₄NF W = OH, X = OH |
| 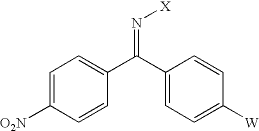 | Fixation of carboxylic acids; cleavage with Hydrazine hydrate (hydrazinolysis); stable in 25 percent strength TFA; W = polymer, X = OH |
| 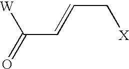 | Fixation of carboxylic acids; cleavage with Pd°/H₂ (catalytic hydrogenation); W = OH, X = OH HYCRAM carrier |
| 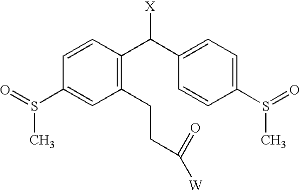 | SCAL linker, fixation of carboxylic acids, cleavage with (EtO)₂P(S)SH/TFA (reductive acidolysis); W = OH, X = NH₂ |
| 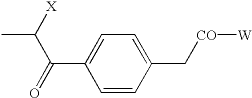 | Fixation of carboxylic acids; cleavage by Photolysis (λ = 350 nm, room Temperature, 72 h); stable in 50 percent Strength TFA, unstable in hydrazine hydrate; W = OH, X = Cl |
| 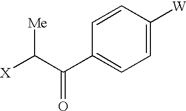 | Fixation of carboxylic acids; cleavage by Photolysis (λ = 350 nm); unstable in Piperidine/DMF; more stable in Piperidine/CH₂Cl₂W = polymer, X = Br |
| 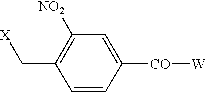 | Fixation of carboxylic acids; cleavage by Photolysis (λ = 350 nm, oxygen-free Under inert gas) X = Hal, OH, NH₂, W = OH |
| W—CO-p-C₆H₄—S—CH₂-X | Rydon linker; P. M. Hardy, H. N. Rydon, R. |
| | C. Thompson, Tetrahedron Lett. 1968, |
| | 2525-2526 |
| | X = OH, W = OH |

The resin polymers which can be used should be insoluble in the liquid phases which are used for the reactions and the isolation of the compounds, substantially inert to the reaction conditions in steps a) to g) and filterable; each resin polymer particle preferably has many binding sites for the respective linkers. Depending on the structure of the selected linkers, structurally completely different resin polymers are possible, for example polystyrene resins, polyamide resins, polydimethylacrylamide resins, modified resins based on the resins mentioned and copolymers. Preferred resins are aminomethylenepolystyrene resins, i.e. aminomethylated polystyrene resins, or alternatively differently modified resins based on polystyrene, for example graft polymers of polystyrene and polyethylene glycol such as those from the series ®TentaGel (Rapp Polymere, Tübingen, Germany), in the form of swellable particles in a particle size range from, for example, 0.01 to 1 mm, preferably 0.05 to 0.5 mm, and a loading of aminomethyl groups from 0.01 to 10 mmol per gram of resin, preferably 0.1 to 2 mmol per gram of resin.

The individual linkers are applied to the resin in a manner known per se; see references mentioned in Tables 1 and 2. All different sorts of techniques can be employed here. Suitable linker components for the combination with the hydroxyl- or aminomethylated polystyrene resins are linkers having carboxylic acid groups which are reacted under the customary conditions for condensations and especially for ester and amide formation reactions. Gentle methods at moderate temperatures are suitable. The reaction can be carried out, for example, in a substantially anhydrous inert organic solvent in the presence of catalysts or customary condensing agents at temperatures from, for example, -30° C. to 200° C., preferably from 0° C. to 150° C., in particular 0° C. to 100° C. Depending on the respective resin, it is also possible to employ aqueous-organic solvents.

The term “inert solvent” refers to solvents which are inert under the reaction conditions in question, but need not be inert under all reaction conditions. For the abovementioned condensation, for example, the following are possible:

ethers such as tert-butyl methyl ether, dimethoxyethane (DME), tetrahydrofuran (THF), diethyl ether, diisopropyl ether,

dipolar aprotic solvents such as dimethylformamide (DMF), N,N-dimethylacetamide (DMA), N-methylpyrrolidone (NMP), acetonitrile,

optionally halogenated aliphatic or aromatic hydrocarbons such as dichloromethane, toluene, o-chlorotoluene, chlorobenzene, or

mixtures of inert solvents.

Suitable condensing means for the preparation of the resin-linker compound (X) from the linker component (XII) and a hydroxymethylene- or aminomethylenepolystyrene resin are customary means such as azeotropic distillation, reaction with activated derivatives of the carboxylic acid such as halides or activated esters. Gentle methods are particularly suitable, such as reaction in the presence of carbodiimides such as dicyclohexylcarbodiimide (DCC) or diisopropylcarbodiimide.

Once the [resin polymer]-linker-X-structures (X) described above have been prepared, they are reacted with compounds of the formula (XI) to give compounds of the formula (II). These are, for example, compounds (X) where X=OH or NH₂ which are reacted with carboxylic acids of the formula (XI) (Y¹=—COOH) to give resin-linked esters or amides (Z=—O—CO— or —NH—CO—).

The optionally substituted (hetero)aromatic adducts of the formula (II) which are accessible in this manner must carry a functional group (S¹) which enables Pd-catalyzed reactions for the synthesis of (hetero)aryl-phosphorus(III) compounds to be carried out (cf. the similar conditions of the Heck reaction; see Lit.; R. F. Heck, Palladium, Reagents in Organic Synthesis, Academic Press 1985).

A central step of the process according to the invention is the synthesis of an arylphosphorus compound (phosphorus(III) compound) on the solid phase (step a) where derivatives of the hypophosphoric acid of the formula (III) are employed under conditions similar to the Heck reaction.

Processes of this type where phosphinates such as H₂PO₂CH₃ or H₂PO₂C₂H₅ are used are known from the literature; see Palladium Reagents and Catalysts, Innovations in Organic Synthesis, Jiro Tsuji, John Wiley & Sons, Chichester England 1995, p. 243 ff, furthermore in Haiyan Lei, Mark S. Stoakes, Kamal P. B. Herath, Jinho Lee and Alan W. Schwabacher, J. Org. Chem. 59 (1994), 4206-4210, furthermore in Haiyan Lei, Mark. S. Stoakes, Alan W. Schwabacher, Synthesis 1992, p. 1255-1260.

Typical examples of a group with which palladium-catalyzed reactions of the abovementioned nature can be carried out are organic halogen compounds, preferably iodides and bromides, but also pseudohalogens such as triflates or tosylates and others; see, for example, similar reactions and reaction conditions in R. F. Heck, Palladium Reagents in Organic Synthesis, Academic Press, 1985. Preferred starting materials for the process according to the invention are iodides, and alternatively bromides. Pseudohalides, for example triflates or tosylates, can be prepared from suitable precursors, for example phenols. This synthesis of pseudohalides can be carried out on solid phase.

Similar to the Heck reaction which is known from the literature, the palladium can be employed in the form of Pd(II) salts, for example bistriphenylphosphane-Pd (II) dichloride, which form a reactive Pd(0) complex in situ. Alternatively, it is possible to employ Pd(0) complexes such as tetrakistriphenylphosphane-Pd inter alia.

The abovementioned process for the palladium-catalyzed introduction of a phosphorus-carbon bond yields compounds of the formula (IV) or, after derivatization of the group E¹ to (E¹)′, compounds of the formula (IV)′ as optionally substituted (hetero)aromatic phosphonous esters, preferably having lower alkyl groups A¹, for example C₁-C₈-alkyl, in particular C₁-C₅-alkyl, in the ester moiety.

A further optional step of the invention comprises the hydrolysis of the phosphonous esters on solid phase to phosphonous acids (V) or (V)′. For this purpose, a strong organic base such as, for example, 1,8-diazabicyclo[5.4.0]undec-7-ene, is reacted in a solvent in which the resin polymer is swellable and which is inert under the reaction conditions, for example in general the solvents mentioned further above, preferably tetrahydrofuran (THF), dioxane or ethylene glycol diethers, with addition of a suitable amount of water, preferably 1 to 100 equivalents, preferably 1 to 10 equivalents, at temperatures from 0 to 100° C., preferably 10 to 50° C. The resulting phosphonous acids can then be reacted on solid phase to give activated esters, for example by reaction with pivaloyl chloride in acetonitrile/pyridine (for literature on similar reactions, see B. C. Froehler; M. D. Matteucci, Tetrahedron Lett. 27 (1986), 469), and these can be reacted with virtually any alcohols R³—OH to give a wide variety of phosphonous esters of the formula (VI) or (VI)′. Further suitable esterification methods for phosphonous acids are described in the following literature references:

Xiadong Cao, A. M. M. Mjalli, Tetrahedron Letters 37 (1996) 6073-6076,

Changzhi Zhang, A. M. M. Mjalli, Tetrahedron Letters 37 (1996) 5457-5460,

In principle, the step of the hydrolysis and the esterification provides access to a wide variety of the radical R³ in formula (I).

The invention in particular also relates to the resin-bound processes for reacting adducts of the formulae (IV) to (VI) or (IV)′ to (VI)′, whose common structural feature is the phosphonous acid or phosphonous monoester group with a series of compounds having functional groups which add to the above-mentioned phosphorus(III) compounds forming phosphorus-carbon bonds (introduction of the radical R²).

It is possible, for example, to activate the compounds mentioned, preferably after reaction of the phosphorus component with silylating agents such as, for example, trimethylsilyl chloride/triethylamine, bistrimethylsilylacetamide, or hexamethyldisilazane or else mixtures of the silylation agents, and to convert them with electrophiles, for example aldehydes, imines, isocyanates or Michael acceptors, into the corresponding products of the formula (VII) and (VIII) or (VIII)′ to (VIII)′; see similar methods for the silylation in:

Kamyar Afarinkia, Charles W. Rees, Tetrahedron 46 (1990) 7175-7196;

E. A. Boyd A. C. Reagan, Tetrahedron Letters 35 (1994), 42234226;

J. K. Thottathil, O. E. Ryono, C. A. Przybyla, J. L. Moniot, R. Neubeck Tetrahedron Lett. 25 (1984) 4741-4744;

O. A. Evans, K. Hurst, J. M. Jakaes, J.Am Chem. Soc. 100 (1978) 3467;

K. Issleib et al., DD-Patent 24 28 10,

Alternatively, the compounds of the formula (IV) to (VI) or (IV)′ to (VI)′ can be reacted with the abovementioned electrophiles under base catalysis. Suitable bases are, in addition to inorganic salts such as, for example, potassium tert-butoxide, in particular organic nitrogen bases such as, for example, triethylamine, 1,8-diazabicyclo[5.4.0]undec-7-ene, N-isopropyl-N-ethylamine and similar compounds (Lit: R. B. Fox, W. J. Bailey, J.Org. Chem. 25 (1960)1447; see also Houben-Weyl, “Methoden der Org. Chemie”, Georg Thieme Verlag 1963, vol. 12/1, “Organische Phosphorverbindungen”).

The selection of the suitable reaction sequence for activating the compounds of the formula (IV) to (VI) or (IV)′ to (VI)′ depends on the specific chemical reactivities of further functional groups in E¹, (E¹)′ or R¹ and gives the person skilled in the art the opportunity to carry out diverse chemical modifications of E¹ or (E¹)′. The resulting phosphinic acid derivatives of the formula (VII) to (VIII) or (VII)′ to (VIII)′ are likewise sufficiently stable to allow diverse chemical modifications of the radicals E¹, (E¹)′ and E². The radicals E¹, (E¹)′ and E² have to be selected in such a way as to make possible a modification of the radicals to the radicals R¹ or R² in formula (I).

In the manner described, it is possible to prepare preferably compounds of the formula (I) in which

R¹ is phenylene which is unsubstituted or substituted by 1 to 4 radicals selected from the group consisting of halogen, alkyl, haloalkyl, alkoxy, haloalkoxy, alkylthio, hydroxyl, amino, nitro, cyano, azido, alkoxycarbonyl, alkylcarbonyl, formyl, carbamoyl, mono- and dialkylaminocarbonyl, acylamino, preferably alkanoylamino having 1 to 6 carbon atoms, mono- and dialkylamino, alkylsulfinyl, haloalkylsulfinyl, alkylsulfonyl and haloalkylsulfonyl, where each substituent may have up to 6 carbon atoms in the alkyl moiety, or is a heteroaromatic radical selected from the group consisting of the 5- or 6-membered ring having in each case 1, 2 or 3 hetero atoms selected from the group consisting of N, O and S, where the radical is unsubstituted or substituted by 1 to 4 radicals selected from the group consisting of halogen, alkyl, haloalkyl, alkoxy, haloalkoxy, alkylthio, hydroxyl, amino, nitro, cyano, azido, alkoxycarbonyl, alkylcarbonyl, formyl, carbamoyl, mono- and dialkylaminocarbonyl, substituted amino such as acylamino, preferably alkanoylamino having 1 to 6 carbon atoms, mono- and dialkylamino, alkylsulfinyl, haloalkylsulfinyl, alkylsulfonyl and haloalkylsulfonyl, and where each substituent may preferably have up to 6 carbon atoms, in particular 4 carbon atoms, in the alkyl moiety, and

R² is hydrogen, an aliphatic hydrocarbon radical which is unsubstituted or substituted and contains, inclusive of substituents, 1 to 30 carbon atoms, preferably 1 to 20 carbon atoms,

R³ is hydrogen or an aliphatic hydrocarbon radical which is unsubstituted or substituted and contains, inclusive of substituents, 1 to 30 carbon atoms, preferably 1 to 20 carbon atoms, or is an aryl or heteroaryl radical which is unsubstituted or substituted and contains, inclusive of substituents, 1 to 30 carbon atoms, preferably alkyl, alkenyl, alkynyl having in each case 1 to 12 carbon atoms where each of the 3 abovementioned radicals is unsubstituted or substituted by one or more radicals selected from the group consisting of halogen, haloalkyl, alkoxy, haloalkoxy, alkylthio, nitro, cyano, alkoxycarbonyl, alkylcarbonyl or unsubstituted or substituted phenyl, where each substituent may have up to 4 carbon atoms in the alkyl moiety

Y is H, COOH, CONH₂, OH, NH₂ or alkylamino.

Of particular interest are compounds (I) where R³═(C₁-C₄)alkyl and Y═COOH.

Preference is given to compounds of the formula (I), or to the preparation thereof according to the invention, in which two or more of the radicals R¹ to R³ and Y have in each case one of the meanings already mentioned or one of the preferred meanings mentioned further below.

The phosphonous acid (ester) group can be derivatized using very different processes with electrophilic reaction partners; for example, in most instances it is possible to react the following classes of substances with compounds of the formula (IV):

1) Aldehydes react to α-hydroxyphosphinic acid derivatives

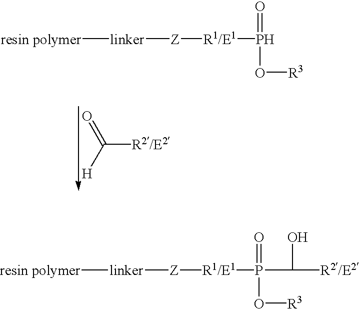

2) Imines react to subst. α-aminophosphinic acid derivatives

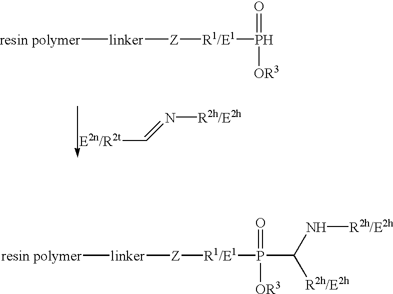

3) Isocyanates react to substituted aminoacylphosphinic acid derivatives

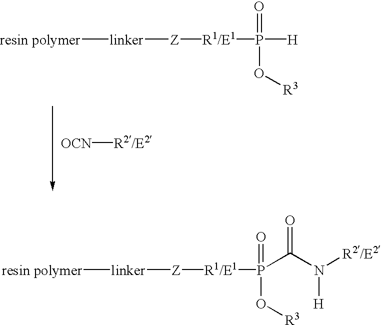

4) Michael acceptors react to substituted ethylphosphinic acid derivatives (V=electronegative group)

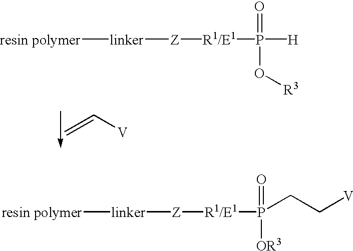

5) Halogen compounds react to phosphinic acids or esters thereof (Arbuzov-type reaction)

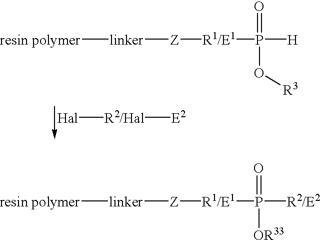

The radicals R²′ and E²′, R²′ and E²″ shown in the formulae of the reaction schemes 1) to 5) are only parts of those radicals R² and E² in the compounds of the formulae (VII) and (VIII) or (VII)′ and (VIII)′ which are introduced at the phosphorus atom in the respective reaction. The remaining parts of the radicals R² and E² lie in the functional groups of the reactants and have been drawn explicitly in the reaction schemes for clarification. Therefore, attention has to be paid to the fact that the radicals R²′ and E²′, R²″ and E²″ are not identical to the radicals R² and E² in the formulae (VII) and (VIII) or (VII)′ and (VIII)′.

For all reaction types 1) to 5), the phosphorus(III) compounds of the formulae (IV) can be deprotonated, for example by adding a suitable base such as, for example, triethylamine, 1,8-diazabicyclo[5.4.0]undec-7-ene and diisopropylethylamine, or they can be activated by silylations and subsequently reacted with the above-mentioned suitably functionalized compounds. Corresponding reactions can also be carried out with the compounds of the formulae (V) and (VI) or (IV)′ to (VI)′.

Suitable reagents for silylating the abovementioned compounds are, for example, trialkylsilyl chlorides/trialkylamines, hexamethyldisilazane, bistrimethylsilylacetamide and other silylating agents known to the person skilled in the art.

Possible interactions between functional groups in the organic radicals E¹, R¹, (E¹)′, E² and R² have to be taken into account in a manner known to the person skilled in the art in the selection of the activation reactions by deprotonation or silylation. There is no general limitation on the radicals in the compounds of the type (IV), (V) or (VI), (VII) or (IV)′, (V)′ or (VI)′.

In general, the types of derivatization reactions or reactions where the radicals E¹ or E² described above are converted into radicals R¹ or R² are known, and most can be applied under similar reaction conditions, some preferred process measures being required by special features of the resin body. Possible interactions between functional groups in the organic radicals E¹ and E² or (E¹)′ and (E²)′ or R¹and R² have to be taken into account in a manner known to the person skilled in the art in the selection of the derivatization reactions. There is no general limitation on the organic radicals in the compounds (VII), (VIII) or (IX) or (VII)′ or (VIII)′.

In the manner described, it is possible to prepare, in an orderly fashion, structure-based substance libraries which are particularly suitable for the systematic examination of the individual compounds it comprises or mixtures thereof for biological or physical-technical properties. Depending on the number of the starting materials of different structure and the number of different reactions and reaction steps, such a substance library can contain from one compound to as many compounds as desired. In particular by the structure of the starting materials, by the reactions and by the reaction sequence, each of the compounds in a substance library is structurally defined. The invention therefore also provides the novel compounds which are contained in the substance libraries according to the invention.

An example of the preparation of a systematic substance library for test compounds is shown in scheme 1 (see next page).

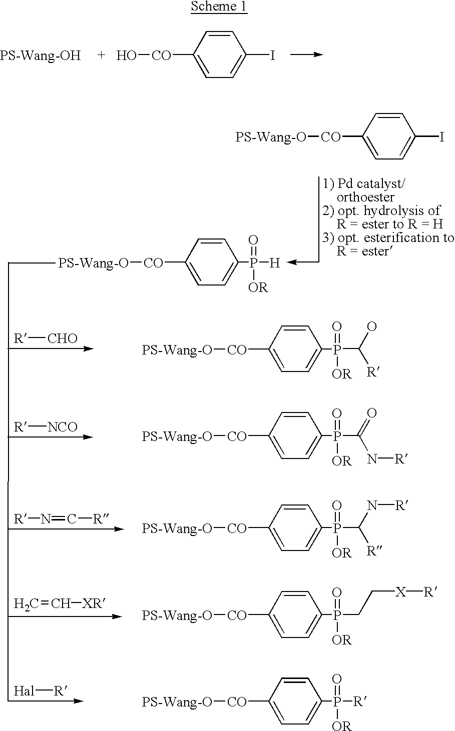

According to Scheme 1, by linking an iodobenzoic acid (here, for example, p-iodobenzoic acid as compound (XI)) to the resin-linker compound (X) (here for example a polystyrene-Wang-linker compound) the p-iodobenzenecarbonyloxy-resin-linker adduct (=compound (II)) is obtained which, with an alkyl orthoformate under Pd catalysis, affords the corresponding benzenephosphonous acid-resin-linker adduct (=compound (IV)). The resin-bound intermediate of the formula (II) according to the invention is subsequently used for preparing corresponding sub-groups of phosphinic ester derivatives of the kind shown by addition and substitution reactions with a series of aldehydes, imines, isocyanates, Michael acceptors and haloorganic compounds (Scheme 1, R=alkyl).

If the phosphonous ester group is hydrolyzed to the compound of the formula (V) prior to the reaction of the compound (IV), a corresponding substance library having resin-bound free phosphonic acids results (Scheme 1, R=H). Likewise, after renewed esterification of the compound (V), for example with benzyl alcohol to the compound of the type (VI), a corresponding substance library having resin-bound free benzyl phosphinates is obtained (Scheme 1, R=CH₂C₆H₅).

In similar reactions using other resin-linker systems according to Table 1, for example using a polystyrene-Rink-amide linker, it is likewise possible to prepare the resin-bound products.

Subsequent to the reactions according to Scheme 1 and similar derivatizations, the resin-bound products (“scaffold”) are advantageously removed by cleavage of the linker-scaffold bond. The cleavage conditions depend on the individual linker; generally, they are known from the literature or can be optimized in preliminary experiments. The cleavage yields the desired synthesis products of the formula (I). In the case of the Wang linker and the Arbuzov reaction according to Scheme 1, bottom row, an alkyl (p-carboxyphenyl)(R′)phosphinate of the formula (Ia)

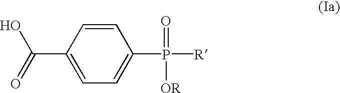

in which R′ is as defined for R² except hydrogen, is obtained as product after cleavage using trifluoroacetic acid.

The process variants described offer a wide choice of structures of the radical R² in compounds of the formula (I). Of particular interest are compounds (I) and the resin-bound intermediates mentioned in which R² is hydrogen or an aliphatic acyclic or cyclic hydrocarbon radical having 1 to 20 carbon atoms or heterocyclyl having 3 to 7 ring atoms and 1, 2 or 3 hetero atoms selected from the group consisting of N, O and S, where the hydrocarbon radical or the heterocyclyl radical is in each case unsubstituted or substituted by one or more radicals selected from the group consisting of halogen, alkoxy, alkenyloxy, alkynyloxy, haloalkoxy, haloalkenyloxy, haloalkynyloxy, alkylthio, amino, nitro, cyano, azido, alkoxycarbonyl, alkenyloxycarbonyl, alkynyloxycarbonyl, alkylcarbonyl, alkenylcarbonyl, alkynylcarbonyl, formyl, carbamoyl, mono- and dialkylaminocarbonyl, acylamino, mono- and dialkylamino, alkylsulfinyl, haloalkylsulfinyl, alkylsulfonyl and haloalkylsulfonyl, unsubstituted and substituted cycloalkyl, unsubstituted and substituted cycloalkenyl, unsubstituted and substituted aryl, unsubstituted and substituted heterocyclyl, unsubstituted and substituted cycloalkoxy, unsubstituted and substituted cycloalkenyloxy, unsubstituted and substituted aryloxy, unsubstituted and substituted heterocyclyloxy, unsubstituted and substituted cycloalkylamino, unsubstituted and substituted cycloalkenylamino, unsubstituted and substituted arylamino, unsubstituted and substituted heterocyclylamino, and in the case of cyclic radicals also alkyl and haloalkyl.

Preference is given to radicals having lower-chain hydrocarbon (alkyl) moieties, for example having 1 to 8 carbon atoms, preferably 1 to 6 carbon atoms, in particular having 1 to 4 carbon atoms.

Preference is also given to compounds (I) and resin-bound intermediates thereof in which

R² is a radical of the formula (R²a), (R²b), (R²c), (R²d) or (R²e),

—CHOH—R* (R²a)

—CO—NH—R* (R²b)

—CHR**—NH—R* (R²c)

—CR^{a}R^{b}—CR^{c}R^{d}—X—R^{e} (R²d)

—R* (R²e)

in which

R* is an aliphatic acyclic or cyclic hydrocarbon radical having 1 to 12 carbon atoms or heterocyclyl having 3 to 6 ring atoms and 1, 2 or 3 hetero atoms selected from the group consisting of N, O and S, where the hydrocarbon radical or the heterocyclyl radical is in each case unsubstituted or substituted by one or more radicals selected from the group consisting of halogen, alkoxy, alkenyloxy, alkynyloxy, haloalkoxy, haloalkenyloxy, haloalkynyloxy, alkylthio, amino, nitro, cyano, azido, alkoxycarbonyl, alkenyloxycarbonyl, alkynyloxycarbonyl, alkylcarbonyl, alkenylcarbonyl, alkynylcarbonyl, formyl, carbamoyl, mono- and dialkylaminocarbonyl, acylamino, preferably alkanoylamino, mono- and dialkylamino, alkylsulfinyl, haloalkylsulfinyl, alkylsulfonyl, haloalkylsulfonyl, unsubstituted and substituted cycloalkyl, unsubstituted and substituted cycloalkenyl, unsubstituted and substituted aryl, unsubstituted and substituted heterocyclyl, unsubstituted and substituted cycloalkoxy, unsubstituted and substituted cycloalkenyloxy, unsubstituted and substituted aryloxy, unsubstituted and substituted heterocyclyloxy, unsubstituted and substituted cycloalkylamino, unsubstituted and substituted cycloalkenylamino, unsubstituted and substituted arylamino, unsubstituted and substituted heterocyclylamino, and in the case of cyclic radicals also alkyl and haloalkyl,

R** is a radical selected from the group of the radicals defined for R* or

R* and R** together are an alkylene bridge which is unsubstituted or substituted by one or more radicals which are, independently of one another, selected from the group of the substituents at the hydrocarbon radical for R*, and R^{a}, R^{b}, R^{c}, R^{d}, R^{e} independently of one another are in each case a radical selected from the group of the radicals defined for R* or

R^{a}, R^{c} or R^{d}, R^{e} or R^{c}, R^{e} in pairs are an alkylene bridge which is unsubstituted or substituted by one or more radicals which are, independently of one another, selected from the group of the substituents at the hydrocarbon radical for R*.

Preference is given to radicals having lower-chain hydrocarbon (alkyl) moieties, for example having 1 to 8 carbon atoms, preferably 1 to 6 carbon atoms, in particular having 1 to 4 carbon atoms.

Preferred substituents of particular interest in the general terms such as “substituted cycloalkyl”, “aryl”, “heterocyclyl” are the preferred substituents mentioned further above in the general definition of substituted radicals.

Further possibilities for variations in the preparation of compounds of the formula (I) result from the possibility to modify the resin-bound intermediates of the formula (II). A derivatization of the compound (II) and its subsequent processing are outlined in an example in Scheme 2 (see next page).

According to Scheme 2, for example 5-iodoanthranilic acid is linked to a resin polymer carrying the Wang linker. 5-Iodoanthranilic acid contains a functional group (amino group) which can be derivatized. Instead of introducing the amino group via anthranilic acid, its preparation is also possible by reduction of a nitro group. Suitable for the reduction are many chemical reducing agents suitable for nitro groups, for example metal salts under acidic conditions, and preference is given to mild reducing agents which can be employed in organic solvents, for example tin dichloride dihydrate —HCl or catalytic reductions.

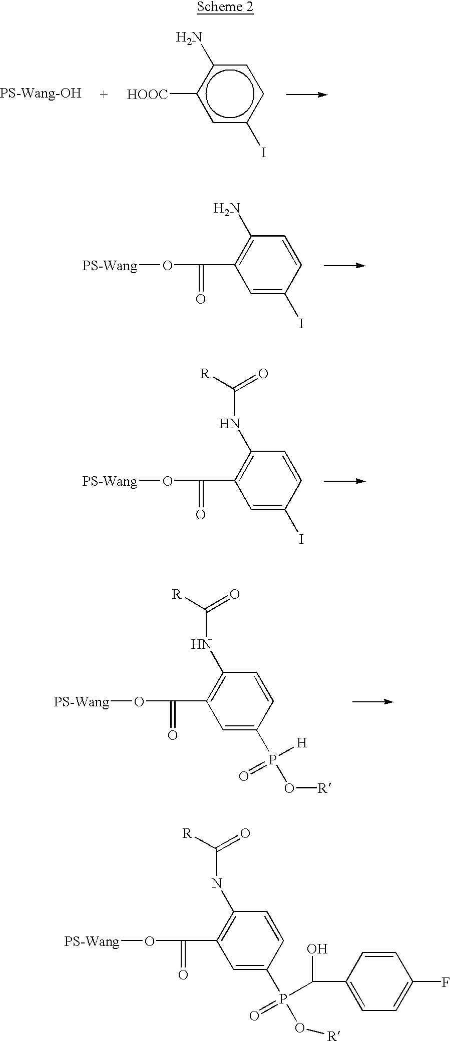

The resulting amino compound can be modified further, for example by (reductive) alkylation or, as shown here, by acylation. The acylation in turn can be carried out successfully with a large number of acylating agents, for example with acyl halides or carboxylic acids, if appropriate with addition of suitable activating reagents such as, for example, triethylamine or carbodiimides. The process according to the invention is subsequently employed in the synthesis of the palladium-catalyzed preparation of the phosphorus-carbon bond. Further derivatization can then be carried out analogously to Scheme 1, for example the reaction with 4-fluorobenzaldehyde to give the last compound in Scheme 2.

### EXAMPLES

Abbreviations:

The commercially available polystyrene-Wang-linker compound 4-hydroxymethylphenyloxymethylpolystyrene resin (Rapp Polymere, Tübingen, Germany) is hereinbelow abbreviated to hydroxy-Wangpolystyrene resin.

| | |
|---|---|
| DMF = | Dimethylformamide |
| THF = | Tetrahydrofuran |
| TFA = | Trifluoroacetic acid |
| TMS = | Trimethylsilane or trimethylsilyl |
| CH₂Cl₂ = | Dichloromethane, Methylene chloride |
| DBU = | 1,8-Diazabicyclo[5.4.0]undec-7-ene |
| DMSO = | Dimethyl sulfoxide |
| min = | minute(s) |
| h = | hour(s) |

% and other quantitative ratios relate to the weight, unless they are specifically otherwise defined in the text.

1) Preparation of the Resin-Linker Compound

4-Iodobenzoyloxy-Wangpolystyrene Resin

500 ml of anhydrous methylene chloride were initially charged together with 12.5 g (15.4 mmol of hydroxyl function) of hydroxy-Wangpolystyrene resin (200-400 mesh, 1.23 mmol of OH per gram of resin). This suspension was admixed with 11.44 g (4.6 mmol) of 4-iodobenzoic acid, 0.83 g (6.55 mmol) of dimethylaminopyridine and 10.5 g (83.1 mmol) of diisopropylcarbodiimide. The suspension was shaken for 3 h and then left to stand for 16 h. The suspension was filtered and the resin polymer was washed repeatedly with DMF (5 times), THF (5 times) and methylene chloride (5 times), in each case a total of 1 l of solvent. The resin polymer was subsequently washed with 400 ml of diethyl ether. The crude product was dried under reduced pressure. Yield of crude product: 15.41 g (82.2%).

2) Preparation of the Resin-Linker Adduct

4-(Ethoxyphosphinoyl)benzoyloxy-Wangpolystyrene Resin (=Ethyl Phosphonite (IVa))

Under an argon atmosphere, 15.00 g (14.4 mmol of iodoaryl function) of 4-iodobenzoyloxy-wangpolystyrene resin were suspended in 250 ml of tetrahydrofuran and admixed with 4.75 g (6.80 mmol) of bis(triphenylphosphane)palladium(II) dichloride and 17.46 g (172 mmol) of anhydrous N-methylmorpholine. To this suspension, a solution which had been prepared as follows was added under an atmosphere of protective gas:

10.44 g (158.2 mmol) of anhydrous hypophosphoric acid, prepared by evaporation of 21 g of a 50% strength aqueous solution of this acid and subsequent drying (1 hour) over 4 A molecular sieves, was stirred in 184.4 g of triethyl orthoformate (1.244 mol) for 2 hours. After the addition, the suspension was immediately heated to 65° C. and kept under reflux for 45 min. The suspension was subsequently cooled under protective gas and the resin polymer was filtered off and washed with 400 ml of a 5% strength acetic acid in tetrahydrofuran and subsequently with 600 ml of tetrahydrofuran, repeatedly with methylene chloride and repeatedly with diethyl ether. The resin was dried under reduced pressure. Yield of crude product: 14.53 g (100.14%).

3) Ethyl P-(4-carboxyphenyl)-P-[(4-nitrophenyl)hydroxymethyl]-phosphinate

300 mg (0.3 mmol) of 4-(ethoxyphosphinoyl)benzoyloxy-Wangpolystyrene resin were suspended in 2 ml of anhydrous methylene chloride. The solution was then cooled to 5° C. and 7.50 ml of a 1 M solution of triethylamine in methylene chloride and then 6.75 ml of a 1 M solution of trimethylsilyl chloride in methylene chloride were added. Within 1 h, the reaction mixture was warmed to room temperature, then filtered under protective gas and subsequently admixed with 6.0 ml of a 1 M solution of 4-nitrobenzaldehyde in methylene chloride. The mixture was shaken at room temperature for 2 h and the resin was filtered off and washed repeatedly with tetrahydrofuran and methylene chloride. The resin was dried under reduced pressure.

The product is subsequently cleaved off by shaking the resin in a 20% strength solution of trifluoroacetic acid in methylene chloride for 1 h. After filtration and concentration of the organic phase, the product is obtained without any further purification in more then 90% purity. Yield: 100 mg (92% of theory).

¹H-NMR (TMS/DMSO d₆): δ (ppm)=1.08 (t, J=6.9 Hz, 1.8H, CH₂—CH₃, diastereomer 1), 1.10 (t, J=6.9 Hz, 1.2H, CH₂—CH₃, diastereomer 2), 3.88 (q, J=6.9 Hz, 2.6H, CH₂—CH₃, diastereomer 1), 3.94 (q, J=6.9 Hz, 1.4H, CH₂—CH₃, diastereomer 2), 5.35 (d, J=12 Hz, 0.6H, CH—OH, diastereomer 1), 5.43 (d, J=16 Hz, 0.4H, CH—OH, diastereomer 2), 6.4 (s br., 1H, COOH), 7.48-7.60 (m, 3H, aromatic H, 7.72-7.83 (m, 3H, aromatic H) 8.0-8.2 (m, 2H, aromatic H), 8.13-8.2 (m, 1H, aromatic H).

4) Ethyl P-(4-carboxyphenyl)-P-[N-isopropyl-1-aminoethyl]phosphinate

1.00 g (1.0 mmol of P—H-functionality) of 4-(ethoxyphosphinoyl)benzoyloxy-Wangpolystyrene resin were preswollen with 2 ml of methylene chloride, then admixed with 2.30 g (22.7 mmol) of triethylamine in 2.0 ml of methylene chloride and subsequently reacted with a solution of 2.24 g (20.7 mmol) of trimethylsilyl chloride. The mixture was shaken at room temperature for 1.5 h, the liquid was filtered off and a solution of 2.55 g (29.9 mmol) of N-isopropylethaneimine in 4 ml of methylene chloride was added. The suspension was shaken at room temperature for 3 h. The suspension was then filtered off and washed repeatedly with methylene chloride, tetrahydrofuran and finally again with methylene chloride and diethyl ether. Yield of crude product: 1.07 g (99%)

The cleavage of 27.3 mg of the crude product with a 50% strength solution of TFA in dichloromethane gave 9.2 mg of the title product (94.6% yield).

¹H-NMR (DMSO-d₆/TMS): δ (ppm) 1.05-1.45 (m, br., 12H, all CH₃), 3.42 (sept., J=7.0 Hz, 0.6H), (CH₃)₂—CH diastereomer 1), 3.60 (sept, J=7.0 Hz, 0.4H, (CH₃)₂—CH diastereomer 2), 3.90 to 4.20 (m br., 3H, CH₂O, P—CH—N), 7.96 (m, 2H, aromatic H), 8.70 (m, 2H, aromatic H), 9.0 (s, br, 2H COOH, NH)

5) 4-(Hydroxyphosphinoyl)benzoyloxy-Wangpolystyrene resin (=Phosphonous acid (Va))

7.00 g (6.90 mmol of P—H functionality) of 4-(ethoxyphosphinoyl)benzoyloxy-Wangpolystyrene resin were initially charged in 70 ml of tetrahydrofuran and admixed with 312 mg (17.3 mmol) of water and 5.28 g (34.7 mmol) of DBU. The suspension was shaken at room temperature for 1 h and filtered and the solid phase was washed with 5% strength acetic acid in THF (5 times 100 ml), with THF (5 times 100 ml), with methylene chloride (5 times 100 ml) and finally with diethyl ether (2 times). The resin polymer was dried under reduced pressure for 12 h. Yield of crude product: 7.57 g (112%) of the title compound.

6) P-(4-Carboxyphenyl)-P-(1-isopropyl-1-hydroxymethyl)phosphinic Acid

50 mg (0.05 mmol of P—H-functionality) of 4-(hydroxyphosphinoyl)benzoyloxy-Wangpolystyrene resin were admixed with 1 ml of methylene chloride. After 30 min, 1.1 ml of a 1 M solution of triethylamine in methylene chloride were added and the mixture was subsequently admixed with 1 ml of a 1 M solution of trimethylsilyl chloride in methylene chloride. The mixture was shaken at room temperature for 30 min and the liquid was filtered off and admixed with 1 ml of a 1 M solution of isobutyraldehyde in methylene chloride and shaken at room temperature for 1 h. The liquid was then filtered off and the resin was washed with THF (10 times) and methylene chloride (10 times). The resin was subsequently treated with 5 ml of 50% strength trifluoroacetic acid in methylene chloride for 30 min and filtered. The filtrate was concentrated.

Yield of crude product: 14 mg of the title compound (108%).

¹H-NMR (DMSO-d₆TMS): δ (ppm)=0.95 (d, J=11 Hz, 3H, CH₃—CH, rotational isomer 1), 0.98 (d, J=11 Hz, 3H, CH₃—CH, rotational isomer 2), 1.95 (sept, J=11 Hz, 1H(CH₃)₂—CH—), 3.50 (t, J=6 Hz, CH—OH), 4.0-5.0 (s br., 3H, COOH, POH and CH—OH), 7.8 (m, 2H, aromatic H), 8.0 (m, 2H, aromatic H)

7) P-(4-Carboxyphenyl)-P-(1-(4-chlorophenyl)-1-hydroxymethyl)-phosphinic Acid

The process was carried out similarly to the process described in Example 6, except that the aldehyde used was 4-chlorobenzaldehyde. Yield of crude product: 17.3 mg of title compound (105.8%).

¹H-NMR (DMSO, TMS): δ (ppm)=4.0 (s, br, 3H, COOH, P—OH, C—OH), 4.93 (d, J=11.2 Hz, 1H, CH—O), 7.24 (m, 4H, aromatic H), 7.72 (m, 2H, aromatic H), 7.98 (m, 2H, aromatic H).

8) P-(4-Carboxyphenyl)-P-(2-ethoxycarbonylethyl)phosphinic Acid

50 mg (0.05 mmol of OH functionality) of 4-(hydroxyphosphinoyl)benzoyloxy-Wangpolystyrene resin were admixed with 1 ml of methylene chloride and then reacted at room temperature for 1 hour with 392 mg (19 mmol) of bistrimethylsilylacetamide dissolved in 2 ml of methylene chloride. The liquid was filtered off and the reaction described above was repeated. The filter resin was then treated with 1 ml of 1 M solution of ethyl acrylate in methylene chloride and stood at room temperature for 16 h. The liquid was filtered off and the resin was washed with THF (10 times) and methylene chloride (10 times). The resin was then reacted with a 50% strength solution of trifluoroacetic acid in methylene chloride for 30 min. Concentration of the filtrate gave the crude product in a purity of more than 90%; yield: 17.5 mg (122%).

¹H-NMR (DMSO-d₆/TMS): δ (ppm)=1.12 (t, 7.2 Hz, 3H, CH₂—CH₃), 2.04 (m, 2H, CH₂—COOEt), 2.38 (m, 2H, CH₂—CH₂COOEt), 3.98 (q, J=7.2 Hz, OCH₂—CH₃), 7.82 (m, 2H, aromatic H), 8.06 (m, 2H, aromatic H).

9) P-(4-Carboxyphenyl)-P-[N-benzyl-1-isopropylaminomethyl]-phosphinic Acid

The process was carried out exactly like the process described in Example 8. Instead of the aldehyde solution, 15 ml of a 0.5 molar solution of N-benzylisopropylmethaneimine in methylene chloride were added and the mixture was stirred at room temperature for 3 h. The liquid was filtered off and the resin was washed with methylene chloride (10 times), THF (10 times) and again with methylene chloride (10 times). The cleavage was carried out by reacting the resin with 3 ml of a 50% strength solution of trifluoroacetic acid in methylene chloride for 30 minutes.

Concentration of the solution gave the crude product in a purity of more than 90%. Yield of the crude product: 22 mg (126%).

¹H-NMR (DMSO-d₆/TMS): δ=0.82 (d, J=8.0 Hz, 3H, CH₃—CH—CH₃), 0.98 (d, J=7.0 Hz, 3H, CH₃—CH—CH₃), 2.10 (m, 1H, (CH₃)₂CH), 3.06 (dd, J_{PH}=12.4 Hz, J_{C—H}=4 Hz, 1H, P—CH—C), 4.34 (AB-spectrum, JHAHB=12.0 Hz, 2H, CH₂—N), 7.4 (m, 5H, aromatic H), 7.82 (m, 2H, aromatic H), 8.06 (m, 2H, aromatic H).

10) 2-Amino-5-iodobenzoyloxy-Wangpolystyrene Resin

Under an atmosphere of protective gas, 15.0 g (18.5 mmol) of hydroxyl-Wangpolystyrene resin (200-400 μm, Rapp Polymere) were suspended in 200 ml of anhydrous methylene chloride. 14.56 g (55 mmol) of iodoanthranilic acid, 12.58 g (99.6 mmol) of diisopropylcarbodiimide and 0.990 g (8.10 mmol) of 4-N,N-dimethylaminopyridine were then added and the suspension was shaken for 24 h. The resin was filtered off and washed 5 times with a total of 2 l of DMF, 5 times with a total of 1 l of THF and repeatedly with a total of 1 l of methylene chloride. The solid was then washed twice with ether and thoroughly dried under reduced pressure. Yield: 18.58 g (94.9%).

11) 2-Isopropylcarbonylamino-5-iodobenzoyloxy-Wangpolystyrene Resin

550 mg (0.52 mmol) of 2-amino-5-iodobenzoyloxy-Wangpolystyrene resin were suspended in 15 ml of anhydrous methylene chloride, cooled to 0° C. and subsequently admixed with 526 mg (5.2 mmol) of triethylamine and 554 mg of isobutyryl chloride. The reaction solution had warmed to room temperature. After 16 h of stirring the solid phase was then filtered off and washed fifteen times with methylene chloride and three times with ether. Yield: 530 mg=90.4%.

12) 5-Ethoxyphosphinoyl-2-isopropylcarbonylaminobenzoyloxy-Wangpolystyrene Resin

0.500 g (0.443 mmol of aryliodide functionality) of 2-isopropylcarbonylamino-5-iodobenzoyloxy-Wangpolystyrene resin were suspended in 5 ml of anhydrous THF under an atmosphere of argon and admixed with 0.538 g (5.30 mmol) of N-methylmorpholine and 0.146 g (0.208 mmol) of bis(triphenylphosphane)palladium(II) dichloride. A solution that had been prepared as follows was subsequently added to this suspension:

0.320 g (4.90 mmol) of crystalline hypophosphoric acid were dried for one hour under an atmosphere of protective gas using 4 Å molecular sieves and stirred with 5.68 g (38.3 mmol) of triethyl orthoformate for 2.5 h. This solution was added to the suspension described above which was then rapidly heated to reflux temperature for 1 h. The reaction mixture was then quickly cooled, washed ten times with a 5% strength solution of acetic acid in tetrahydrofuran, ten times with methylene chloride and finally three times with diethyl ether. The resulting resin was dried under reduced pressure; yield of crude product: 458 mg (94.5%).

A trial cleavage using 50 mg of resin (1 h, 20% strength trifluoroacetic acid/methylene chloride) gave 17.3 mg (105%) of crude product.

¹H-NMR (DMSO-d₆/TMS): δ (ppm)=1.08 (d, J=8.0 Hz, 6H, (CH₃)₂CH), 1.23 (t, J=5.6 Hz, 3H, CH₂—CH₃), 2.60 (sept, J=8.0 Hz, 1H, (CH₃)CH), 4.07 (m, 2H, P—O—CH₂), 7.55 (d, J=576 Hz, 1H, P—H), 7.83 (m, 1H, aromatic H), 8.35 (m,1H, aromatic H), 8.73 (m, 1H, aromatic H).

13) Ethyl P-(3-carboxy-4-isopropylcarbonylaminophenyl)-P-(pyrid-4-yl-hydroxymethyl)phosphinate

50 mg (0.046 mmol of P—H functionality) of 5-ethoxyphosphinoyl-2-isopropylcarbonylaminobenzoyloxy-Wangpolystyrene resin were suspended in 1 ml of methylene chloride and admixed with 1.1 ml of a 1 M solution of triethylamine in methylene chloride and 1.0 ml of a 1 M solution of trimethylsilyl chloride in methylene chloride. The solution was shaken for 30 min and then filtered off and admixed with 1 ml of a 1 M solution of nicotinaldehyde in methylene chloride. After 1 h of reaction at room temperature, the reaction solution was filtered off, the resin was washed ten times with tetrahydrofuran and methylene chloride and the product was liberated from the resin by cleaving for 30 min using 50% strength trifluoroacetic acid in methylene chloride. The crude product was concentrated using a rotary evaporator and obtained in a purity of more then 95% as a glass-like material. Yield: 21 mg (96%).

¹H-NMR (DMSO-d₆/TMS): δ (ppm)=1.13 (t, 6.4 Hz, 2H, O—CH₂—CH₃ diastereomer 1), 1.18 (d, 7 Hz, 6H, (CH₃)₂CH), 1.23 (t, 6.4 Hz, 1.8H, OCH₂CH₃, diastereomer 2), 2.60 (sept, J=7 Hz, 1H, (CH₃)₂—CH), 3.89 (quart, J=8.0 Hz, 0.8H, CH₂—CH₃, diastereomer 1), 4.06 (quart, J=8.0H₂, 1.2H, CH₂—CH₃, diastereomer 2), 5.43 (d, J=12.8 Hz, 0.4H, P—CH diastereomer 1), 5.60 (d, J=16.0 Hz, 0.6H, P—CH diastereomer 2), 7.97 (m, 2H, aromatic H, 7.80 (m, 1H, aromatic H), 8.28 (m, 1H, aromatic H), 8.64 (m, 1H, aromatic H), 8.76 (m, 2H, aromatic H), 11.4 (s,1H, COOH).

14) N-(4-Iodobenzoylamino)-Rink-amide-polystyrene Resin

The Fmoc group was removed from 10.00 g (7.8 mmol of NH functionality) of Fmoc-Rink-amide-polystyrene resin by shaking the resin with 100 ml of a 20% strength piperidine/DMF solution for 30 minutes, followed by filtration. The process was repeated once more and the resin was then washed thoroughly with DMF. The resin was resuspended in 120 ml of DMF and admixed with 6.15 g (25.0 mmol) of 4-iodobenzoic acid, 3.20 g (25.0 mmol) of diisopropylcarbodiimide and 3.33 g (25.0 mmol) of 1-hydroxybenzotriazol. The mixture was shaken for 4 hours and the resin was then filtered off and subsequently washed five times each with DMF, THF and methylene chloride. The resin was dried under reduced pressure. Yield of crude product: 9.80 g (99% of theory) of the title compound.

15) N-[4-(ethoxyphosphinoyl)benzoyl]-Rink-amide-polystyrene Resin

Under an atmosphere of argon, 1.00 g (0.77 mmol of iodoaryl function) of N-(4-iodobenzoyl)-Rink-amide-polystyrene resin was suspended in 10 ml of anhydrous tetrahydrofuran and admixed with 258 mg (0.370 mmol) of bistriphenylphosphanepalladium(II) dichloride and 1.00 ml (9.09 mmol, 0.92 g) of anhydrous N-methylmorpholine. Under an atmosphere of protective gas, this suspension was admixed with a solution which had been prepared as follows: 0.55 g (8.3 mmol) of anhydrous hypophosphoric acid, prepared by evaporation of 1.1 g of a 50% strength aqueous solution of this acid and drying for one hour over a 4 Å molecular sieve, and 10.0 g (67.5 mmol) of triethyl orthoformate were stirred together for 2 h.

After the addition of the resulting solution, the suspension was immediately heated to 65° C. and kept at reflux for 45 min. The mixture was subsequently cooled under protective gas and the resin polymer was filtered off and washed with 50 ml of a 5% strength acetic acid in tetrahydrofuran and subsequently with 150 ml of tetrahydrofuran, repeatedly with methylene chloride and repeatedly with diethyl ether. The resin was dried under reduced pressure. Yield: 0.950 g (97% of theory) of the title compound.

16) Ethyl P-(4-aminocarbonylphenyl)-P-[(4-fluoro-phenyl)hydroxymethyl]phosphinate

50 mg (0.063 mmol of P—H functionality) of N-[4-(ethoxyphosphinoyl)benzoyl]-Rink-amide-polystyrene resin were admixed with 1.5 ml (1.5 mmol) of a 1 M solution of 1,8-diazobicyclo[5.4.0]undec-7-ene (DBU) in dichloromethane. After 15 min, the solution was filtered off and the resin was admixed with 1.5 ml of a 1 M solution of 4-fluorobenzaldehyde in dichloromethane. After 1 h of shaking at room temperature, the resin was filtered off and washed repeatedly with tetrahydrofuran and dichloromethane. The resin was dried under reduced pressure. The product was subsequently cleaved off by shaking the resin in a 20% strength solution of trifluoroacetic acid in dichloromethane for 1 h. Filtration and concentration of the organic phase gave the product without any further purification in a purity of more than 90%. Yield: 11 mg (82% of theory).

¹H-NMR (DMSO-d₆/TMS): δ (ppm)=1.18 (t, J=6.9 Hz, 3H, CH₂—CH₃, diastereomer 1), 1.19 (t, J=6.9 Hz, 3H, CH₂—CH₃, diastereomer 2), 3.85 (q, J=6.9 Hz, 2H, CH₂—CH₃, diastereomer 1), 3.91 (q, J=6.9 Hz, 2H, CH₂—CH₃, diastereomer2), 5.10 (d, J=12 Hz, 1H, CH—OH, diastereomer 1), 5.22 (d, J=15 Hz, 1H, CH—OH, diastereomer 2), 7.10 (t, J=6 Hz, 2H, aromatic H), 7.31 (m, 2H, aromatic H), 7.55 (s, brd, 1H, NH), 7.7 (m, 2H, aromatic H), 7.91 (m, 2H, aromatic H), 8.12 (s, brd, 1H, NH).

17) Synthesis of a Substance Library According to Scheme 1 and Scheme 2

The systematic synthesis of substance libraries is possible using the experimental procedure described in the text above. Starting from 5-iodoanthranilic acid which is bound to a polymer via the carboxylic acid function and the Wang linker, a substance library was synthesized by the following stepwise reactions:

The compound of the formula

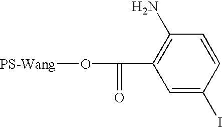

was reacted with the acylating agents acetyl chloride, propionyl chloride, isopropylcarbonyl chloride and cyclohexylcarbonyl chloride to give the four different N-acyl compounds. The palladium-catalyzed reaction with bis(triphenylphosphane)palladium(II) dichloride with hypophosphoric acid and triethyl orthoformate similar to Example 2 gave the four ethyl phosphonites according to the formula (IV), and subsequent hydrolysis gave the four phosphonous acids according to the formula (V). The eight resulting phosphorus-containing resin-linker adducts according to (IV) and (V) were in each case reacted with 10 different aldehydes and 10 different isocyanates according to Tables A and B:

TABLE A
Aldehydes of the formula (A1)
H—CO—R* (A1)

| No. | R* |
|---|---|
| 1 | Phenyl |
| 2 | 4-Chlorophenyl |
| 3 | 4-Methoxyphenyl |
| 4 | 3,4-Dichlorophenyl |
| 5 | 2-Chlorophenyl |
| 6 | 2,4-Dichlorophenyl |
| 7 | 2-Fluorophenyl |
| 8 | 3-Bromo-4-fluorophenyl |
| 9 | 2-Methoxyphenyl |
| 10 | 2,6-Dichlorophenyl |

TABLE B
Isocyanates of the formula (A2)
O═C═N—R* (A2)

| No. | R* |
|---|---|
| 1 | 4-Chlorophenyl |
| 2 | 3,4-Dichlorophenyl |
| 3 | 3,5-Dichlorophenyl |
| 4 | 2,4-Dichlorophenyl |
| 5 | 4-Bromophenyl |
| 6 | 4-Isopropylphenyl |
| 7 | 1-Naphthyl |
| 8 | 3-(2,2-Dichloro-1,1-difluoroethoxy)phenyl |
| 9 | 3-Methoxycarbonylphenyl |
| 10 | 2-Butoxyphenyl |

The substance library which was obtained, comprising 160 resin-linker adducts of the formula (IX)

[resin polymer]-[linker-Z-R¹—P(R²)(═O)—O—R³]ₙ (IX)

in which R² is a radical of the formulae (R²a) or (R²b),

—CHOH—R* (R²a)

—CO—NH—R* (R²b)

in which R* is as defined by Tables A or B, and the remaining radicals and groups are as defined above,

was cleaved using 20% strength trifluoroacetic acid in methylene chloride, and a further substance library comprising 160 compounds of the formula (Ib)

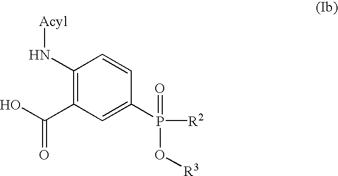

in which

Acyl=Acetyl, propionyl, isopropylcarbonyl or cyclohexylcarbonyl,

R²=(R²a) or (R²b), which are the 20 different radicals mentioned,

R³=H or ethyl and

Y=COOH

was obtained.

1. A process using intermediates which are linked to a resin polymer for preparing chemical compounds of the formula (I) 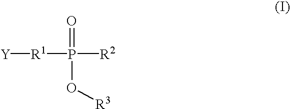
in which

| | |
|---|---|
| R¹ | is an unsubstituted or substituted aromatic or heteroaromatic radical, |
| R² | is hydrogen or an organic radical which may be linked to the rest of |
| | the compound of formula (I) via hetero atoms, |
| R³ | is hydrogen or an organic radical which is attached via a carbon atom |
| | and |
| Y | is the functional group which is formed at the molecule of the |
| | formula (I) after the compound (I) has been cleaved off from the |
| | resin polymer, |

which comprises
a) reacting a resin-linker adduct of the formula (II)
[resin polymer]-[linker-Z-E¹-S¹]ₙ (II)
in which

| | |
|---|---|
| [resin polymer] | is the radical of a resin which, in the resin-linker |
| | compound (II), is connected via n binding sites with the n |
| | groups of the formula [linker-Z-E¹-S¹], |
| linker | is in each case an organic linker, |
| Z | is a linker-specific functional group or bond which, after |
| | cleavage of the compound (I) from the resin polymer- |
| | linker radical, gives rise to the group Y in formula (I), |
| E¹ | is defined as R¹ in formula (I) or is a radical which is |
| | suitable for preparing R¹ in compound (I), |
| S¹ | is a functional group suitable for palladium-catalyzed |
| | substitutions analogous to the Heck reaction, |
| n | is the number of the functional groups [linker-Z-E-S] at |
| | the resin, which depends on the molecular weight of the |
| | resin polymer and is greater than or equal to 1, |

in the presence of a suitable palladium catalyst with a compound selected from the group of the phosphinates (derivatives of hypophosphoric acid) of the formula (III)
A¹-O—(PHO)A* (III)
in which

| | |
|---|---|
| A¹ | is hydrogen or an organic radical, |
| A* | is a group which can be removed hydrolytically or after an inter- |
| | mediate reaction, |

with substitution of the group S¹ to give a resin-bound compound of the formula (IV)
[resin polymer]-[linker-Z-E¹-P(H)(═O)—O-A¹]ₙ (IV)
in which A¹ is as defined in formula (III), and
b) derivatizing, if appropriate, the compound (IV) in one or more further reaction steps at the organic radical E¹ to give the radical (E¹)′, thus yielding one or more resin-bound intermediates of the formula (IV)′
[resin polymer]-[linker-Z-(E¹)′—P(H)(═O)—O-A¹]ₙ (IV)′
in which A¹ is as defined in formula (III), and
c) hydrolyzing, if appropriate, the compound of the formula (IV) or (IV)′ from step a) or b) to give a compound (V) or (V)′ suitable for the resin-bound synthesis
[resin polymer]-[linker-Z-E¹-P(H)(═O)—OH]ₙ (V)
[resin polymer]-[linker-Z-(E¹)′-P(H)(═O)—OH]ₙ (V)′
and
d) esterifying, if appropriate, the compound (V) or (V)′ obtained according to c) to give the compound of the formula (VI) or (VI)′
[resin polymer]-[linker-Z-E¹-P(H)(═O)—O—R³]ₙ (VI)
[resin polymer]-[linker-Z-(E¹)′-P(H)(═O)—O—R³]ₙ (VI)′
in which
R³ is defined as R³ in formula (I), but is not hydrogen, and
e) reacting, if appropriate, a compound (IV), (V) or (VI) or (IV)′, (V)′ or (VI)′ obtained according to a), b), c) or d), whose common structural feature is the phosphonous acid or phosphonous ester group, forming a phosphorus-carbon bond, to give compounds of the formulae (VII) or (VIII) or (VII)′ or (VIII)′
[resin polymer]-[linker-Z-E¹-P(R²)(═O)—O-A⁴]ₙ (VII)
[resin polymer]-[linker-Z-(E¹)′—P(R²)(═O)—O-A⁴] (VII)′
[resin polymer]-[linker-Z-E¹-P(E²)(═O)—O-A⁴]ₙ (VIII)
[resin polymer]-[linker-Z-(E¹)′—P(E²)(═O)—O—A⁴]ₙ (VIII)′
in which
R² is as defined in formula (I),
E² is an organic radical which can be derivatized to the radical R²,
A⁴=A¹, H or R³, and
f) modifying the compounds obtained according to the abovementioned steps if required at the radicals E¹, (E¹)′, E² and A⁴ in such a manner that the resin-bound compound of the formula (IX) is obtained
[resin polymer]-[linker-Z-R¹—P(R²)(═O)—O—R³]ₙ (IX)
in which R¹, R², R³ are as defined in formula (I), and
g) cleaving the compound of the formula (I) from the resin-linker adduct of the formula (IX),
where in the formulae (IV) to (IX) and (IV)′ to (VIII)′ the radicals [resin polymer], linker, Z are as defined in formula (II) and E¹ or (E¹)′ in the formulae (V) to (VIII) or (V)′ to (VIII)′ are as defined in formula (IV) or (IV)′.

2. The process as claimed in claim 1, wherein
R¹ is phenylene which is unsubstituted or substituted by 1 to 4 radicals selected from the group consisting of halogen, alkyl, haloalkyl, alkoxy, haloalkoxy, alkylthio, hydroxyl, amino, nitro, cyano, azido, alkoxycarbonyl, alkylcarbonyl, formyl, carbamoyl, mono- and dialkylaminocarbonyl, acylamino, mono- and dialkylamino, alkylsulfinyl, haloalkylsulfinyl, alkylsulfonyl and haloalkylsulfonyl, where each substituent may have up to 6 carbon atoms in the alkyl moiety,
or is a heteroaromatic radical selected from the group consisting of the 5- or 6-membered ring having in each case 1, 2 or 3 hetero atoms selected from the group consisting of N, O and S, where the radical is unsubstituted or substituted by 1 to 4 radicals selected from the group consisting of halogen, alkyl, haloalkyl, alkoxy, haloalkoxy, alkylthio, hydroxyl, amino, nitro, cyano, azido, alkoxycarbonyl, alkylcarbonyl, formyl, carbamoyl, mono- and dialkylaminocarbonyl, substituted amino such as acylamino, mono- and dialkylamino, alkylsulfinyl, haloalkylsulfinyl, alkylsulfonyl and haloalkylsulfonyl, and where each substituent may have up to 6 carbon atoms in the alkyl moiety, and
R² is hydrogen, an aliphatic hydrocarbon radical which is unsubstituted or substituted and contains, inclusive of substituents, 1 to 30 carbon atoms,
R³ is hydrogen or an aliphatic hydrocarbon radical which is unsubstituted or substituted and contains, inclusive of substituents, 1 to 30 carbon atoms, or is an aryl or heteroaryl radical which is unsubstituted or substituted and contains, inclusive of substituents, 1 to 30 carbon atoms, and
Y is H, COOH, CONH₂, OH, NH₂ or alkylamino.

3. The process as claimed in claim 1, wherein
R² is hydrogen or an aliphatic acyclic or cyclic hydrocarbon radical having 1 to 20 carbon atoms or heterocyclyl having 3 to 7 ring atoms and 1, 2 or 3 hetero atoms selected from the group consisting of N, O and S, where the hydrocarbon radical or the heterocyclyl radical is in each case unsubstituted or substituted by one or more radicals selected from the group consisting of halogen, alkoxy, alkenyloxy, alkynyloxy, haloalkoxy, haloalkenyloxy, haloalkynyloxy, alkylthio, amino, nitro, cyano, azido, alkoxycarbonyl, alkenyloxycarbonyl, alkynyloxycarbonyl, alkylcarbonyl, alkenylcarbonyl, alkynylcarbonyl, formyl, carbamoyl, mono- and dialkylaminocarbonyl, acylamino, mono- and dialkylamino, alkylsulfinyl, haloalkylsulfinyl, alkylsulfonyl, haloalkylsulfonyl, unsubstituted and substituted cycloalkyl, unsubstituted and substituted cycloalkenyl, unsubstituted and substituted aryl, unsubstituted and substituted heterocyclyl, unsubstituted and substituted cycloalkoxy, unsubstituted and substituted cycloalkenyloxy, unsubstituted and substituted aryloxy, unsubstituted and substituted heterocyclyloxy, unsubstituted and substituted cycloalkylamino, unsubstituted and substituted cycloalkenylamino, unsubstituted and substituted arylamino, unsubstituted and substituted heterocyclylamino, and in the case of cyclic radicals also alkyl and haloalkyl.

4. The process as claimed in claim 3, wherein
R² is a radical of the formula (R²a), (R²b), (R²c), (R²d) or (R²e),
—CHOH—R* (R²a)
—CO—NH—R* (R²b)
—CHR**—NH—R* (R²c)
—Cr^{a}R^{b}—CR^{c}R^{d}—X—R^{e} (R²d)
—R*(R²e)
in which
R* is an aliphatic acyclic or cyclic hydrocarbon radical having 1 to 12 carbon atoms or heterocyclyl having 3 to 6 ring atoms and 1, 2 or 3 hetero atoms selected from the group consisting of N, O and S, where the hydrocarbon radical or the heterocyclyl radical is in each case unsubstituted or substituted by one or more radicals selected from the group consisting of halogen, alkoxy, alkenyloxy, alkynyloxy, haloalkoxy, haloalkenyloxy, haloalkynyloxy, alkylthio, amino, nitro, cyano, azido, alkoxycarbonyl, alkenyloxycarbonyl, alkynyloxycarbonyl, alkylcarbonyl, alkenylcarbonyl, alkynylcarbonyl, formyl, carbamoyl, mono- and dialkylaminocarbonyl, acylamino, mono- and dialkylamino, alkylsulfinyl, haloalkylsulfinyl, alkylsulfonyl, haloalkylsulfonyl, unsubstituted and substituted cycloalkyl, unsubstituted and substituted cycloalkenyl, unsubstituted and substituted aryl, unsubstituted and substituted heterocyclyl, unsubstituted and substituted cycloalkoxy, unsubstituted and substituted cycloalkenyloxy, unsubstituted and substituted aryloxy, unsubstituted and substituted heterocyclyloxy, unsubstituted and substituted cycloalkylamino, unsubstituted and substituted cycloalkenylamino, unsubstituted and substituted arylamino, unsubstituted and substituted heterocyclylamino, and in the case of cyclic radicals also alkyl and haloalkyl,
R** is a radical selected from the group of the radicals defined for R* or
R* and R** together are an alkylene bridge which is unsubstituted or substituted by one or more radicals which are, independently of one another, selected from the group of the substituents at the hydrocarbon radical for R*, and
R^{a}, R^{b}, R^{c}, R^{d}, R^{e} independently of one another are in each case a radical selected from the group of the radicals defined for R* or
R^{a}, R^{c} or R^{d}, R^{e} or R^{c}, R^{e} in pairs are an alkylene bridge which is unsubstituted or substituted by one or more radicals which are, independently of one another, selected from the group of the substituents at the hydrocarbon radical for R*.

5. The process as claimed in claim 1, wherein
R³═(C₁-C₄)alkyl and Y═COOH.

6. The process as claimed in claim 1, wherein

| | |
|---|---|
| Z | is a group of the formula —O—CO— or —NH—CO— and |
| S¹ | is an iodine atom. |

7. A process for preparing a compound of the formula (IV)
[resin polymer]-[linker-Z-E¹-P(H)(═O)—O-A¹]ₙ (IV)
in which A¹, [resin polymer], linker, Z, E¹ and n are as defined in claim 1 and
which comprises reacting a resin-linker adduct of the formula (II)
[resin polymer]-[linker-Z-E¹-S¹]ₙ (II)
in which
[resin polymer], linker, Z, E¹ and n are as defined in formula (IV) and S¹ is a functional group suitable for palladium-catalyzed substitutions analogous to the Heck reaction,
in the presence of a suitable palladium catalyst with a compound selected from the group of the phosphinates (derivatives of the hypophosphoric acid) of the formula (III)
A¹-O—(PHO)A* (III)
in which
A¹ is hydrogen or an organic radical,
A* is a group which can be removed hydrolytically or after an intermediate reaction,
with substitution of the group S¹ to give the compound of the formula (IV).

8. A process for preparing a compound of the formula (VII)
[resin polymer]-[linker-Z-E¹-P(E²)(═O)—O-A⁴]ₙ (VII)
in which A⁴, [resin polymer], linker, Z, E¹, E² and n are as defined in claim 1,
which comprises reacting a resin-linker adduct of the formula (IV)
[resin polymer]-[linker-Z-E¹-P(H)(═O)—O-A⁴]ₙ (IV)
in which A⁴, [resin polymer], linker, Z, E¹ and n are as defined in formula (VII),
with an electrophile, forming a phosphorus-carbon bond, to give compounds of the formulae (VII).

9. A process for preparing compounds of the formula (I) 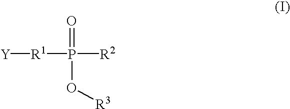
which comprises cleaving the compound of the formula (I) from a resin-linker adduct of the formula (IX)
[resin polymer]-[linker-Z-R¹—P(R²)(═O)—O—R³]ₙ (IX)
where in the formulae (I) and (IX) the radicals [resin polymer], linker, Z, Y, R¹, R², R³ and the number n are as defined in claim 1.

10. A compound of the formula (II), (IV), (IV)′, (V), (V)′, (VI), (VI)′, (VII), (VII)′, (VIII), (VIII)′ and (IX), as defined in claim 1.

11. A compound library, comprising compounds of the formula (II), (IV), (IV)′, (V), (V)′, (VI), (VI)′, (VII), (VII)′, (VIII), (VIII)′ or (IX), as defined in claim 1, or mixtures thereof.

12. A compound library, comprising compounds of the formula (I) as defined in claim 1.

13. A compound library, comprising compounds of the formula (I), (IV), (IV)′, (V), (V)′, (VI), (VI)′, (VII), (VII)′, (VIII), (VIII)′ or (IX), prepared according to a process as claimed in claim 1.

14. A method for testing compounds for biological activity as medicament or crop protection agent which comprises testing compounds from a compound library as claimed in claim 11.

15. A method for testing compounds for biological activity as medicament or crop protection agent which comprises testing compounds from a compound library as defined in claim 13.
